(19) United States
(12) Patent Application Publication
(10) Pub. No.: US 2011/0229842 A1
(43) Pub. Date: Sep. 22, 2011

(54) METHOD AND DEVICE FOR THREE-DIMENSIONAL MEASUREMENT OF A DENTAL MODEL

(76) Inventors: Uwe Bielfeldt, Bad Soden (DE); Reiner Engelmohr, Egelsbach (DE); Dirk Markgraf, Kronberg/Taunus (DE); Michael Stolper, Eschborn (DE)

(21) Appl. No.: 13/117,758

(22) Filed: May 27, 2011

Related U.S. Application Data
(63) Continuation-in-part of application No. PCT/IB2009/055423, filed on Nov. 30, 2009.

(30) Foreign Application Priority Data
Nov. 29, 2008 (EP) 08 020 791.3

(51) Int. Cl. A61B 6/14; G06G 7/48

(52) U.S. Cl. 433/29; 433/215; 703/6

(57) ABSTRACT

A new three-dimensional modelling system for dental surfaces has presently been discovered. The modelling system may include a light source and a camera. Once a three-dimensional model is produced, it can be used, for example, to assess the performance of tooth cleaning products.

### CROSS REFERENCE TO RELATED APPLICATION

This application is a continuation-in-part of copending International Application No. PCT/IB2009/055423 filed Nov. 30, 2009 and designating the United States which claims priority to EP Application No. 08020791.3 filed on Nov. 29, 2008, the disclosures of which are incorporated herein by reference.

### FIELD OF THE INVENTION

The invention herein relates to methods and devices for three-dimensional measurement of dental models.

### BACKGROUND OF THE INVENTION

The determination of cleaning performance is an important parameter in the development of dental cleaning products, such as toothbrushes, dental floss, toothpaste, etc. This may be evaluated in so-called clinical tests using test subjects as well as with artificial laboratory methods. The latter have the advantage that they allow a much more rapid assessment of the cleaning performance of a cleaning system. To do so, dental models or dentition casts, to which plaque substitutes have been applied, are used. The cleaning performance is evaluated on the basis of the plaque substitutes remaining on the dental surface and/or dentition surface after a cleaning procedure.

Projections of the dental surfaces be evaluating may be used for detecting residues of plaque substitutes. Projection planes here are the external and internal (buccal and lingual) dental surfaces and the chewing (occlusal) surface. One disadvantage of these methods is that surfaces and/or parts of surfaces of the teeth that are not parallel to the plane of projection, e.g., surfaces of the interdental spaces and at the root of the tooth are not reproduced in their actual size, which can falsify the result of the evaluation. As such, there is a need for improved methods of evaluating the effectiveness of cleaning products.

### SUMMARY OF THE INVENTION

In one embodiment, the present invention is directed to a method for evaluating cleaning performance of a dental cleaning product, comprising: preparing a three-dimensional model of a tooth surface prior to cleaning, preparing a three dimensional model of a tooth surface after cleaning, and comparing the three dimensions models of the tooth surfaces prior to and after cleaning.

In an additional embodiment, the present invention is directed to a method for making a three-dimensional model of a surface of a dental model, comprising: projecting light onto the tooth surface and moving the light on at least a portion of the surface of the dental model, taking a first photograph of at least a portion of the tooth surface at a first triangulation angle, taking a second photograph of at least a portion of the tooth surface at a second triangulation angle, and deriving two dimensions of the three-dimensional model by triangulation based on the first and second photographs, and deriving a third dimension from the movement of the light on at least a to portion of the surface of the dental model.

In another embodiment, the present invention is directed to a device for three-dimensional measurement of a dental model, comprising at least two split-beam measuring devices for generating two measuring surfaces of the dental model, wherein each of the split-beam measuring devices comprises a first camera system, a second camera system, and a light source; wherein an optical axis of the first camera system is inclined at a first triangulation angle with respect to a plane of a beam of light from the light source; wherein an optical axis of the second camera system is inclined by a second triangulation angle with respect to a plane of a beam of light; from the light source; wherein the first triangulation angle is different from the second triangulation angle; wherein the split-beam measuring devices are each arranged at a distance from the dental model, so that four different measuring surfaces of the dental model can be generated, and wherein the dental model and the split-beam measuring devices are arranged so they are movable relative to one another.

These and other embodiments will be better understood in light of the description below.

### BRIEF DESCRIPTION OF THE DRAWINGS

Additional features, possible applications, and advantages of the invention can be derived from the following description of exemplary embodiments of the invention, some of which are depicted in the figures:

FIG. 1 shows a schematic perspective diagram of one embodiment of the inventive device;

FIG. 2 shows a side perspective view of an arrangement of the individual elements of a split-beam measurement device according to one embodiment of the present invention;

FIG. 3 shows an overhead view of the individual elements of the split-beam measurement device according to one embodiment of the invention;

FIG. 4 shows a perspective view of dental model tooth with plaque substitute residues on its surface, according to one embodiment; and

FIG. 5 shows a perspective view of a dental model tooth with plaque substitute residues on an interdental area as well as its surface, according to one embodiment.

### DETAILED DESCRIPTION OF THE INVENTION

The efficacy of a current or proposed tooth cleaning product is often tested by evaluating the product's performance in areas like plaque removal, decrease in gingivitis, etc. Evaluation of to products can take place on the teeth of test subjects or on dental models. When using dental models for plaque removal evaluation, plaque is added to the model with the use of a plaque substitute and the efficacy is measured based on the ability of the product to remove the plaque substitute from the dental model. For plaque removal, some tests were proficient only at determining plaque removal on some portions of a tooth and not proficient at determining the efficacy on all portions of a tooth. This was due to distortions of the measurement on all areas of the tooth not parallel to the measuring device, like the interdental area. It has presently been discovered that the use of a three-dimensional dental model measuring system, like the split beam method, will allow for a more reliable evaluation of the cleaning efficacy of a tooth cleaning product, as it allows for the determination of plaque layer thickness and thus gives a more accurate assessment of the product's performance than previous methods.

In the split-beam method, at least one beam of light is projected onto the dental model surface and moves thereon, whereby a first photograph of the beam of light is created at a first triangulation angle (α₁) to the projection axis of the beam of light. A second photograph of the beam of light is created at a second triangulation angle (α₂) to the projection axis of the beam of light, whereby the first triangulation angle (α₁) is different from the second triangulation angle (α₂). Two dimensions of the dental model are derived from the beam of light in the photographs by means of triangulation.

The third dimension of the dental model is derived from the movement of the beam of light on the surface of the dental model. In one embodiment, the movement of the beam of light on the surface of the dental model is accomplished by rotation of the dental model. Rotating the dental model itself, allows for more easily reproducible photographs which can lead to even more accurate product comparison. In another embodiment, the light and/or camera are rotated around the dental model. Thus, the movement of the light can be accomplished by moving the model, a light source, a camera, or a combination thereof.

Thus, the space coordinates of the surface of the dental model are derived from the photographs and the movement of the beam of light, and a three-dimensional model for display on a display device is generated from the space coordinates. The three-dimensional model preferably describes a height profile of the dental model.

In one embodiment, the intensity distribution of the beam of light on the surface of the dental model can be determined from the photographs. Further, a measure of the reflectivity of the surface of the dental model can then be derived from this. The values for the reflectivity may to be assigned to a plaque substitute residue, whereby the assignment of a reflectivity value to plaque substitute residues and/or to the concentration of plaque substitute residues is made on the basis of one or more reflectivity threshold values, and whereby the plaque substitute residues and/or the concentration of plaque substitute residues may be displayed on the three-dimensional model. The surfaces of the plaque substitute residues on the three-dimensional model can be determined by means of numerical methods. Plaque and/or plaque substitute residues on the dental model can thus be detected in an efficient manner.

The thickness of the layer of plaque substitute residues may be determined from the difference in the height profiles of the three-dimensional model with plaque substitute residues and of the three-dimensional model without residues of plaque substitute.

Furthermore, a device for three-dimensional measurement of a dental model is provided. FIG. 1 shows a schematic perspective diagram of one embodiment of the inventive device for three-dimensional measurement of a dental model. Measurement with respect to only one tooth is shown for simplicity, but the same principal could be applied to multiple teeth.

In FIG. 1, two split-beam measurement devices 10, 10′, each of which has a light source, for example, a laser, 30, 30′ and two camera systems 20, 21 and/or 20′, 21′ are essentially arranged on two opposing sides of the tooth 5 and/or the dental model. The split-beam measurement devices 10, 10′ are arranged in such a way that they simultaneously image the tooth surface (buccal, lingual and occlusal) from two obliquely opposite views.

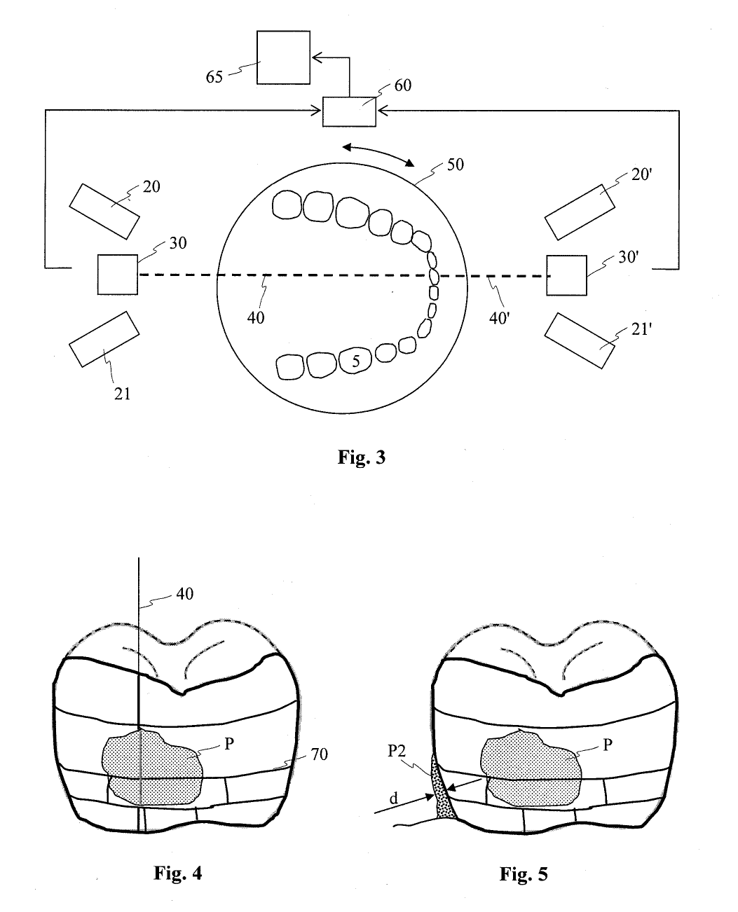

A beam of light 40, 40′, from a laser, for example, is projected onto at least a portion of the surfaces of the dental model by light sources 30, 30′. The beam of light is picked up by the respective camera systems 20, 21 and/or 20′, 21′ and analyzed by an image analyzer unit 60 (shown in FIG. 3). The camera systems and/or the optical axes of the lenses of the camera systems are preferably arranged at a certain angle α₁ and α₂ to the beam of light 40, 40′ and/or at a certain angle α₁ and α₂ to the plane of the surface over which the beam of light 40, 40′ passes. The border of the area over which the beam of light 40 passes is labelled with reference numerals 40a and 40b in FIG. 1.

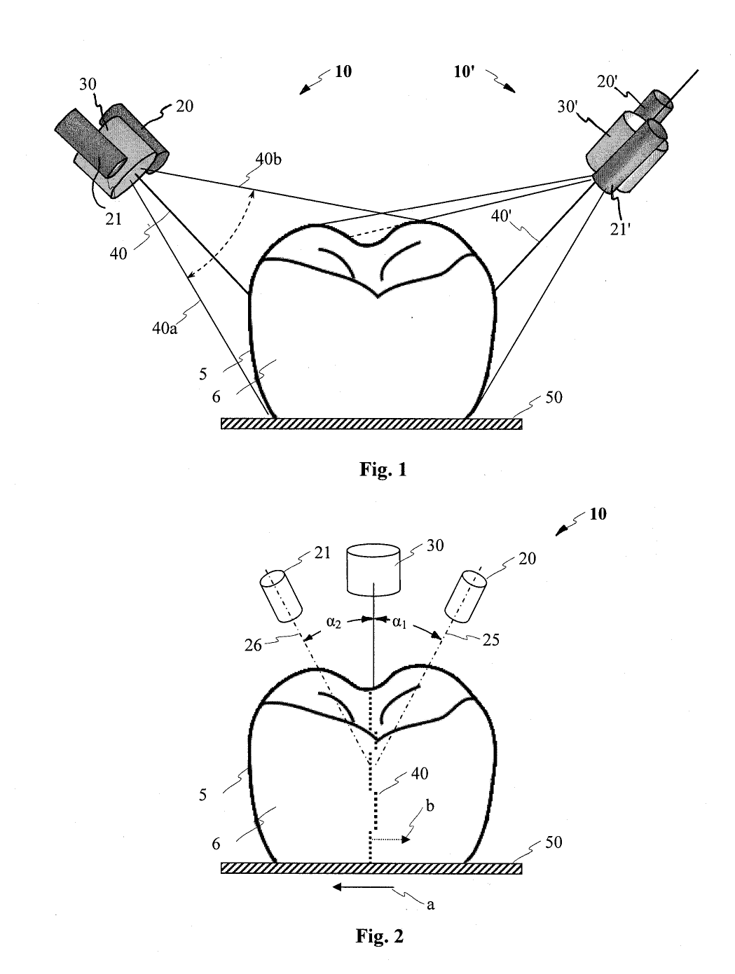

As shown in FIG. 2, the beam of light 40 generated by the laser light source 30 is projected onto the surface of the dental model as a beam of light and/or a split-beam. The camera systems 20, 21 detect a beam of light having a certain offset on the surface being recorded due to its inclination relative to the laser beam of light (as illustrated by the dotted-line beam of light in FIG. 2). The height information required to generate a three-dimensional model can be derived from this to offset by means of known triangulation methods. The height resolution depends on the angles α₁ and/or α₂. The larger the angles α₁ and/or α₂, the greater is the height resolution because the offset of the beam of light is measurable with a greater resolution.

The tooth 5 of the dental model can be arranged on a rotatable disk 50. By rotating the disk 50, the tooth is moved into the measurement volume of the stationary split-beam measurement devices. It is thus possible to detect an entire tooth and/or an entire dental model in three dimensions with a single recording operation. Furthermore, by adjusting the rotational speed, the resolution of the photograph can be increased or reduced in the horizontal direction. Additionally, in another embodiment, all or a portion of the split beam measuring devices could be rotated around the stationary dental model.

By rotating the disk 50 in the direction of the arrow a (as shown on FIG. 2), the dental model arranged on the disk is also rotated in this direction. The beam of light and/or split-beam 40 imaged on the surface of the dental model moves in the direction of the arrow b. The rate of rotation enters into the measurement result together with the acquisition frequency (frequency at which the measurement surfaces are recorded by the camera systems) of the camera systems. The scanning grid therefore depends on the rate of rotation of the dental model on the rotating disk 50 and the acquisition frequency of the camera systems.

With known pixel resolution of the camera systems, the rate of rotation of the rotating disk may be adapted in such a way that preferably a sampling grid with the same resolution in width and height is achieved.

The use of a rotating disk for fixed mounting of the dental model has the advantage of producing more reproducible scanning operations because the positions of the split-beam measuring device can remain fixed in space. Through position pins and/or fastenings arranged on the rotating disk, it is possible to secure a dental model on the rotating disk with accurate positioning.

The camera systems 20, 21 and/or their lenses are inclined at a certain angle α₁ and/or α₂ relative to the laser beam of light 40, where preferably α₂=−αa₁. Due to this arrangement of two camera systems opposite the laser beam of light, it is also possible to detect and record undercuts on the side faces of the tooth (as indicated by the beam shown with a dotted line in FIG .1). The areas that are concealed from one camera system 20 are then visible to the other camera system 21 and can be recorded.

In order for the camera systems of the two split-beam measuring devices 10, 10′ to be able to to differentiate, and/or not record, the beam of light from the first light source 30 from the beam of light of the other light source 30′, the light sources may be operated in such a way that each generates a beam of light of a different frequency. For example, in another embodiment, the camera systems may be equipped with a corresponding filter, which filters out the beam of light of the other split-beam measuring device on the basis of its frequency.

In one embodiment, the camera system may compromise a matrix camera. In another embodiment, telecentric lenses may be provided. In addition, special collimators and microline lenses adapted to generating the beam of light may be arranged on the camera systems in further embodiments.

A three-dimensional model of the dentition and/or the surface of the dentition is generated from the measurements provided by the split-beam measuring devices to the image analyzer unit 60 by using a triangulation method. In addition, the surfaces of the dental model showing residues of plaque are also imaged on the three-dimensional model generated in this way (see also the description of FIG. 4). By measuring the dental model both before and after plaque application and again after applicator of the test product, the thickness of the plaque substitute residues present on the dental model can be determined. The three dimensional model(s) can be displayed on a display device 65 (shown in FIG. 3).

As shown in FIG. 3, the split beam method may also be used to image multiple teeth, here a mandible. The mandible 5 is arranged here on the rotating disk 50.

In this embodiment, a split-beam measuring device is arranged on both sides of the mandible. The split-beam measuring device arranged on the left side of the mandible records the interior (lingual) surface of the dentition in the position of the mandible illustrated in FIG. 3, while at the same time the split-beam measuring device arranged on the right side of the mandible records the exterior (buccal) surface of the dentition. In an arrangement of the split-beam measuring devices obliquely above the dental model (as shown in FIG. 1), the chewing (occlusal) surfaces are recorded by both split-beam measuring devices.

Further to this embodiment, the split-beam measuring devices are coupled to an image analyzer unit 60. The image analyzer unit 60 generates a three-dimensional model of the dental model 5 by means of triangulation methods using the measurement surfaces recorded. The three-dimensional model may then be displayed as needed on a display device 65. In one embodiment, the display device 65 is coupled to the image analyzer unit 60. In addition, the three-dimensional model may also be stored for further processing. Likewise, the measurement surfaces recorded may be saved to allow analysis, e.g., generation of a 3D model, at a later point in time.

FIG. 4 shows a representation of a partial view of a three-dimensional model of a tooth from the front (buccally) which could have been generated with the help of the inventive device. An area P in which residues of a plaque substitute substance are found on the dental model after a cleaning procedure are visibly displayed on the dental surface. The information for the display of plaque substitute residues on the dental surface is obtained according to this invention by the fact that the split-beam measuring devices and/or an image analyzer unit coupled to the split-beam measuring devices determines the intensity distribution of the beam of light 40 on the dental surface. The intensity distribution of the beam of light is different in the area of the plaque substitute residue from the intensity distribution on the clean tooth surface.

A measure of the reflectivity of the surface of the tooth and/or of the dental model is derived from the intensity distribution of the beam of light 40. Taking into account one or more reflectivity threshold values, the distribution and/or concentration of plaque substitute residues on the dental surface is determined and displayed on the three-dimensional dental model. With the help of the reflectivity threshold values, a determination is made about whether or not these are plaque substitute residues. The reflectivity threshold values are preferably adjustable. The areas of the plaque substitute residues on the surface of the tooth are ascertained by means of numerical methods.

For better comparability of different cleaning procedures, e.g., with the help of different tooth cleaning products on a dental model, the surface of the teeth can be divided into cells 70, also known as grids. The number and distribution of the cells on the surface of the tooth may depend on various known plaque indices such as the Quigley-Hein index. The classification of the dental surface in the corresponding cells is performed manually or automatically by the image analyzer unit 60. The cell area may be determined by numerical methods.

The degree of plaque substitute residues in the respective cell is assigned to each cell. The degree of plaque substitute residues may be expressed as the percentage of the cell area or as the absolute area, e.g., in mm² The allocation is preferably made automatically. The comparison of various cleaning procedures on a dental model may be performed then on the basis of the degree of plaque substitute residues in the respective cells. The absolute area of plaque substitute residues on the whole or within a cell can be determined by means of known numerical methods.

Preferably the three-dimensional model together with the cells and the degree of plaque substitute residues can be saved, so that cleaning procedures at different points in time can be compared with one another.

To further improve the relevance of the measurement results with regard to the cleaning performance, the inventive method proposes determining the layer thickness of the plaque substitute residues remaining on the dental surface of the dental model after a cleaning procedure. Thus together with the degree of plaque substitute residues in a cell, an even more differentiated comparison of the cleaning performance of different dental cleaning products may be obtained.

FIG. 5 shows a front view of a representation of a three-dimensional model of a tooth generated with the help of the inventive device as seen from the front (buccally) with plaque substitute residues P and P2. The plaque substitute residues P2 are shown in cross section to illustrate a layer thickness measurement of plaque substitute residues in the interdental area. The difference in the surface area of the tooth from the surface area of the plaque substitute residue P2 forms the thickness d of the plaque substitute residue P2.

To measure the layer thickness of plaque substitute residues on the dental surface, the height information on the dental surface as determined by means of the split-beam method and/or the triangulation method is used. To do so, in a first step a three-dimensional model of a tooth model or a dental model without plaque substitute residues is generated. This three-dimensional model serves as a reference model for the determination of the thickness of plaque substitute residues. The dental model is provided with plaque substitute residues and is then subjected to a cleaning procedure.

In another step, a second three-dimensional model is generated for the cleaned tooth model. By comparing the height information of the reference model with the second three-dimensional model, the thickness of the plaque substitute residues remaining on the surface of the tooth model and/or dental model is ascertained. The height information differs essentially at the locations where the plaque substitute residues are found. The thickness of the plaque substitute residues is then calculated by forming the difference in the height information.

To avoid any inconsistencies which could arise due to differences in measurements where there are no plaque substitute residues, it is possible to limit the evaluation to only those areas where plaque substitute residues are in fact located. These relevant areas may be ascertained with the method already described above on the basis of the intensity measurement of the beam of light. This has the advantage that differences in height information which may occur, e.g., due to non-identical arrangement of the dental model will not be detected as the layer thickness of a plaque substitute residue.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A method for evaluating cleaning performance of a dental cleaning product, comprising:
a) preparing a three-dimensional model of a tooth surface prior to cleaning,
b) preparing a three dimensional model of a tooth surface after cleaning, and
c) comparing the three dimensional models of the tooth surfaces prior to and after cleaning.

2) The method of claim 1, wherein the tooth surface is that of a dental model.

3) The method of claim 2, wherein preparing the three dimensional model of the tooth surface prior to cleaning, comprises:
a) projecting light onto at least a portion of the tooth surface and moving the light on at least a portion of the surface of the dental model,
b) detecting the location of at least a portion of the light on the tooth surface at a first triangulation angle,
c) detecting the location of at least a portion of the light on the tooth surface at a second triangulation angle, and
d) deriving two dimensions of the three-dimensional model by triangulation, and
e) deriving a third dimension from the movement of the light on at least a portion of the surface of the dental model.

4) The method of claim 3, wherein the movement of the light across the surface of the dental model is created by the movement of the dental model, movement of a light source, movement of a camera, or a combination thereof.

5) The method of claim 1, wherein at least a portion of the tooth surface prior to cleaning comprises a known amount of plaque residue.

6) The method of claim 5, wherein the known amount of plaque residue is determined by:
a) making a height profile with a three-dimensional model of the tooth surface prior to application of a plaque residue,
b) making a height profile of a three-dimensional model of the tooth surface after plaque residue application, and
c) comparing the height profile of the tooth surface prior to application of the plaque residue with the height profile of the tooth surface after application of the plaque residue.

7) The method of claim 1, wherein the comparison process comprises comparing a height profile of the tooth surface prior to cleaning to a height profile of the tooth surface after cleaning.

8) A method for making a three-dimensional model of a tooth surface, comprising:
a) projecting light onto at least a portion of the tooth surface,
b) detecting the location of at least a portion of the light on the tooth surface at a first triangulation angle,
c) detecting the location of at least a portion of the light on the tooth surface at a second triangulation angle, and
d) deriving two dimensions of the three-dimensional model by triangulation based on the first and second photographs, and
e) deriving a third dimension from the movement of the light on at least a portion of the tooth surface.

9) The method of claim 8, wherein the light comes from a split-beam light source.

10) The method of claim 8, wherein the light for the first triangulation angle comes from a first light source and the light for the second triangulation angle comes from a second light source.

11) The method of claim 10, wherein a camera performs the act of detecting.

12) The method of claim 11, wherein the movement of the light across the surface of the dental model is created by the movement of the dental model, movement of a light source, movement of a camera, or a combination thereof.

13) The method of claim 12, wherein space coordinates of the tooth surface of the dental model are derived from the photographs and the movement of the light, and a three-dimensional model is generated from the space coordinates, such that the three-dimensional model describes a height profile of the dental model.

14) The method of claim 13, wherein the model is displayed on a display device.

15) A device for three-dimensional measurement of a dental surface, comprising at least two split-beam measuring devices for generating two measuring surfaces of the dental surface, wherein each of the split-beam measuring devices comprises a first camera system, a second camera system, and a light source; wherein an optical axis of the first camera system is inclined at a first triangulation angle with respect to a plane of a beam of light from the light source; wherein an optical axis of the second camera system is inclined by a second triangulation angle with respect to a plane of a beam of light; from the light source; wherein the first triangulation angle is different from the second triangulation angle;
wherein the split-beam measuring devices are each arranged at a distance from the dental surface, so that four different measuring surfaces of the dental surface can be generated, and wherein the dental surface and the split-beam measuring devices are arranged so at least one is moveable relative to the other.

16) The device of claim 15, wherein the first triangulation angle of the first camera system corresponds to the negative second triangulation angle of the second camera system.

17) The device of claim 15, further comprising a rotating device to hold the dental surface.

18) The device of claim 15, wherein the camera systems have telecentric lenses.

19) The device of claim 15, wherein the beams of light are formed by a laser light source comprising a semiconductor laser having collimators and microline lenses.

20) The device of claim 15, further comprising an image analyzing unit connected to the split-beam measuring devices for generating a three-dimensional surface of the dental surface that can be displayed on a display device.
